(19) United States
(12) Patent Application Publication
(10) Pub. No.: US 2014/0169163 A1
(43) Pub. Date: Jun. 19, 2014

(54) SYSTEMS AND METHODS FOR COMMUNICATION CHANNEL CAPACITY CHANGE DETECTION

(71) Applicant: GENERAL ELECTRIC COMPANY, Schenectady, NY (US)

(72) Inventors: Stephen Francis Bush, Latham, NY (US); Michael Joseph Dell'Anno, Clifton Park, NY (US)

(73) Assignee: General Electric Company, Schenectady, NY (US)

(21) Appl. No.: 13/718,678

(22) Filed: Dec. 18, 2012

Related U.S. Application Data
(60) Provisional application No. 61/736,980, filed on Dec. 13, 2012.

(51) Int. Cl. H04L 12/56

(52) U.S. Cl. CPC H04L 47/12
USPC 370/230

(57) ABSTRACT

Systems and methods for communication channel capacity detection are provided. One method includes monitoring a bandwidth over time of a channel communicatively coupling a plurality of medical devices at a first location with a second location remote from the first location and determining when a channel bandwidth of the channel exceeds a defined threshold value using the monitored bandwidth. The method also includes transmitting control signals from the second location to the plurality of devices at the first location to adjust a transmission rate of medical data from the plurality of medical devices to the second location and limiting a rate of transmission of the control signals to the plurality of medical devices based on a probability that the transmission of the control signals causes the channel bandwidth to exceed the defined threshold value.

### CROSS REFERENCE TO RELATED APPLICATIONS

This application claims priority to and the benefit of the filing date of U.S. Provisional Application No. 61/736,980 filed Dec. 13, 2012, the subject matter of which is herein incorporated by reference in its entirety.

### BACKGROUND

Remote health care services, such as performing diagnostic imaging or monitoring in remote locations that otherwise may not have adequate health care facilities, are increasing. The remote health care practice area is growing, due in part to cost reduction, faster diagnosis and the overall efficiency provided by a partial decentralization of health care dispensaries.

In remote health care, a patient may be examined by a remote health care practitioner (RHCP) in a medical dispensary or monitored in a location (e.g., patient's home) remote from a major medical center such as a hospital. For example, a patient may be monitored at a location remote from a specialist, which may include the use of multiple medical devices monitoring the patient at the same time. Accordingly, multiple sources of medical data may be communicated.

In remote locations (e.g., developing countries), the medical data is often communicated over a constrained channel, which is often a channel with low bandwidth (e.g., 2G cellular bandwidth or less) and typically having widely varying channel capacity over time, which may cause a varying Quality of Service (QoS). As a result of the use of the constrained channel to communicate the medical data, diagnostically relevant or diagnostically important information may be delayed or there may be a reduction in the quality of service of the channel, which may include a feedback delay. Accordingly, a delay in diagnosis or treatment, annoyance and aggravation to the RHCP, and in some cases misdiagnosis may result. Also, if the effect of the varying QoS on the communications from the remote location to the specialist is not recognized, characterized, and compensated for, in some instances the overall process is less efficient.

### SUMMARY

In one embodiment, a method for controlling transmission of medical data is provided. The method includes monitoring a bandwidth over time of a channel communicatively coupling a plurality of medical devices at a first location with a second location remote from the first location and determining when a channel bandwidth of the channel exceeds a defined threshold value using the monitored bandwidth. The method also includes transmitting control signals from the second location to the plurality of devices at the first location to adjust a transmission rate of medical data from the plurality of medical devices to the second location and limiting a rate of transmission of the control signals to the plurality of medical devices based on a probability that the transmission of the control signals causes the channel bandwidth to exceed the defined threshold value.

In another embodiment, a medical data communication system is provided that includes a plurality of medical devices at one location configured to acquire medical data for a patient, a transceiver coupled to the plurality of medical devices and a workstation at a location remote from the location of the plurality of medical devices. The medical data communication system also includes a transceiver coupled to the workstation, wherein the transceivers coupled to the plurality of medical devices and the workstation form a communication link therebetween. The medical data communication system further includes a channel capacity monitoring unit at the location of the workstation and configured to monitor a bandwidth over time of a channel of the communication link, determine when a channel bandwidth of the communication link exceeds a defined threshold value using the monitored bandwidth, transmit control signals to the plurality devices to adjust a transmission rate of medical data from the plurality of medical devices, and limit a rate of transmission of the control signals to the plurality of medical devices based on a probability that the transmission of the control signals causes the bandwidth of the communication link to exceed the defined threshold value.

### BRIEF DESCRIPTION OF THE DRAWINGS

FIG. 1 is a schematic block diagram of a medical data communication system formed in accordance with an embodiment.

FIG. 2 is a diagram illustrating a communication link in accordance with various embodiments.

FIG. 3 is a graph of channel bandwidth over time measured in accordance with various embodiments.

FIG. 4 is a diagram of a user interface in accordance with an embodiment.

FIG. 5 is a flowchart of method for channel capacity change detection and transmission rate control in accordance with various embodiments.

### DETAILED DESCRIPTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers, circuits or memories) may be implemented in a single piece of hardware or multiple pieces of hardware. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide communication of medical data, such as monitoring data, which may include medical images. The communication channel(s) in some embodiments are constrained having a dynamic effective bandwidth, which in some embodiments are channels having a low bandwidth and widely varying channel capacity over time. In some embodiments, the constrained communication channel(s) may take the form of a dial up, DSL, cable, 2G, 3G, 4G cellular, power line carrier, radio, satellite, fiber or any type of connection (or lower bandwidth connection) that is constrained with respect to the data being transmitted and the required maximum latency for the desired use.

For example, various embodiments provide a method for optimizing the transmission of medical data from multiple sources over a communication channel that is constrained and having a capacity that is randomly changing. At least one technical effect of various embodiments is increased efficiency or optimized control of multiple sources of medical data, such as video, ultrasound data, blood pressure data, diagnostic audio, electrocardiogram (ECG) data, etc., over a common constrained channel.

In particular, various embodiments provide methods and systems for controlling the rates of data traffic sources from multiple medical devices communicating over a single constrained communication channel such that the desired quality of service for each source is maintained over a communication channel with a changing capacity. Generally, a receiver transmits feedback control messages to the medical data traffic sources indicating at what rate these sources should transmit, given that the channel capacity may change in a random manner during operation. It should be noted that if control messages are sent too frequently, the downstream control channel may become congested and thus increase feedback delay. In addition, the received quality of service may change rapidly, which may reduce the quality of experience for the remote user. However, if the feedback is too slow, the channel capacity may not be optimally utilized by the sources, such that either the combined source load will exceed the channel capacity causing delay and reduced quality of service or the channel may be underutilized.

Various embodiments determine a feedback rate based on the changing conditions or expected change in the channel capacity. The data communication may include, for example, communication of medical data from a plurality of medical devices at one location (e.g., a patient monitoring examination site) to another location (e.g., a hospital remote from the examination site) over one or more constrained communication channels. In one embodiment, for example, a monitoring and/or continuous remote health care practitioner (RHCP) to specialist channel bandwidth feedback rate is provided, such as between a plurality of medical devices monitoring a patient at a location remote from a health care facility and having communication channels that are constrained with a capacity or effective bandwidth that is randomly changing. Thus, various embodiments control the communication of medical data from one or more medical devices over one or more communication channels, which in some embodiments are constrained channels.

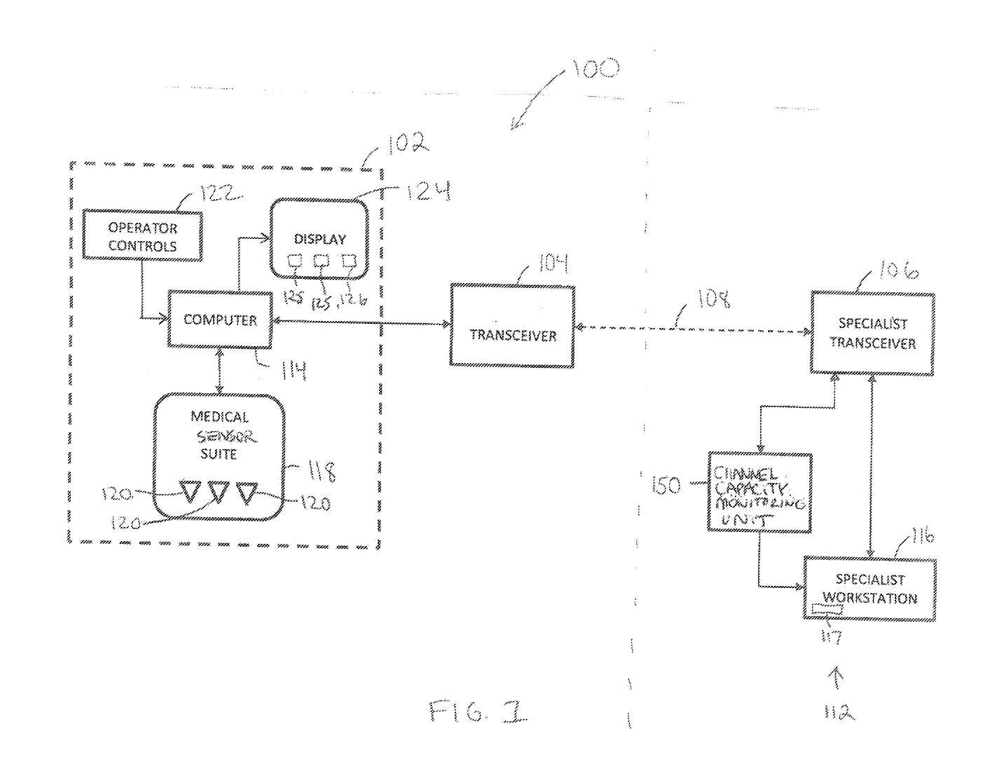

FIG. 1 is a schematic block diagram of a data communication system 100 for communicating medical data in accordance with various embodiments. The medical data communication system 100 is generally configured to acquire medical data (e.g., monitoring or image data), such as patient monitoring information (e.g., blood pressure measurements, ECG, ultrasound imagery, etc.) at a patient location, which may include in some instances a remote health care practitioner (RHCP) and transmit that medical data to, for example, a remotely located specialist for viewing, analysis, treatment and/or consultation. The medical data communication system 100 includes a patient location 102 (e.g., remote dispensary or patient's home) where a patient is being monitored and that allows acquisition of medical data remote from a medical care facility. The patient location 102 may also include an interface for a user or operator, such as the RHCP. It should be noted that although various embodiments are described in connection with communicating certain types of medical data, the various embodiments may be used to communication other types of medical and non-medical data, such as other types of medical images and other physiological data or waveforms, as well as other data.

The system 100 includes a transceiver 104 at the patient location 102 that communicates with a remote transceiver, which in the illustrated embodiment is a specialist transceiver 106, namely a transceiver at a location of a specialist. The transceivers 104, 106 communicate over or form a communication link 108, which may include one or more communication channels (e.g., constrained cellular network communication channels), which in some embodiments have a low bandwidth and a varying or randomly changing effective bandwidth. Accordingly, the communication link 108 provides bi-directional or two-way communication between the patient location 102 and a second location 112 (also referred to as the specialist location 112), which may be a specialist location remote therefrom (e.g., miles away), respectively, in one embodiment.

With respect to the patient location 102 where the medical data is acquired and optionally processed (or partially processed), a processor, which is illustrated as a computer 114, may be coupled to a medical sensor suite 118. In some embodiments, a single computer 114 is coupled to a plurality of medical devices 120 of the medical sensor suite 118. In other embodiments, separate computers 114 may be coupled to the medical devices 120. Additionally, the computer 114 may be integrated or form part of the medical sensor suite 118 (e.g., embodied a processor of the medical devices 120) or may be separate therefrom.

The computer 114 allows communication between the medical devices 120 and a workstation at the second location 112, illustrated as a specialist workstation 116, via the specialist transceiver 106. It should be noted that the transceiver 104 and the specialist transceiver 106 may form part of or be separate from the medical devices 120 and the specialist workstation 116, respectively. It also should be noted that the workstation 116 may be any type of workstation (or electronic tablet device, notebook computer, cellular phone, etc.) usable by different types of operators.

The medical devices 120 may be removably and/or operatively coupled to an interface (now shown) of the computer 114 to allow communication therebetween. The medical sensor suite 118 may include a plurality of different types or kinds of medical devices 120, such as a plurality of different types of medical monitoring devices or imaging devices or probes that may be used for different monitoring and imaging applications (e.g., physiological monitoring).

The computer 114 is also optionally coupled to a user input 122 (also referred to as operator controls 122) that includes one or more user controls (e.g., keyboard, mouse and/or touchpad, touch-screen of a tablet device) for interfacing or interacting with the medical devices 120. Again, a separate user input 122 may be provided in connection with each of the medical devices 120.

The computer 114 is also coupled to a display 124, which may be configured to display medical data 125 or images 126, which may include displaying information from all of the medical devices 120 on a single display or on multiple displays 124 (which may be separately connected to the medical devices 120). The user input 122 may allow a user (e.g., RHCP or patient) to control the display of the medical data 125 or images 126 on the display 124, for example, by controlling the particular display settings. The user input 122 may also allow a user to control the acquisition of the medical or image data.

In operation, data acquired by the medical devices 120 at the patient location 102 is accessible and may be communicated between the patient location 102 and the second location 112 using the transceivers 104, 106. It should be noted that the transceivers 104, 106 may be configured to communicate using any suitable communication protocol, which in various embodiments is a lower bandwidth wireless communication protocol, such as cellular 2 G communication (or power line carrier) protocols or lower that forms a constrained channel as described herein. Using this arrangement, data from the medical devices 120 at the patient location 102 may be transmitted to a specialist at the specialist workstation 116 and data sent from the specialist may be received at the patient location 102.

At the second location 112, which in one embodiment may be a hospital or health care facility having a specialist located there, a channel capacity monitoring unit 150 is configured to monitor a plurality of channel bandwidth samples for data received from the first location 102, via the transceivers 104, 106 and control the transmission rate of the medical devices 120, for example, by transmitting control messages to the medical devices to adjust the transmission rate of one or more of the medical devices 120 as described in more detail. In various embodiments, the channel capacity monitoring unit 150 is a module or controller, which may be implemented in hardware, software, or a combination thereof. The channel capacity monitoring unit 150 is located proximate the specialist workstation 116, which in some embodiments forms part of the specialist workstation 116 or may be a module operatively coupled to the specialist workstation 116. The specialist workstation 116 may be a data server where multiple workstations may be connected and interacting with the computer 114 at the patient location 102.

In various embodiments, the channel capacity monitoring unit 150 is configured to maintain a sliding time window of the last N channel bandwidth samples. For example, the channel capacity monitoring unit 150 may use the channel bandwidth samples to provide feedback control to the medical devices 120 to control the rate of transmission of data from the medical devices 120. For example, the channel capacity monitoring unit (CCMU) 150 computes an adjustment (increase or decrease) to be made to a quality of service (QoS) of the data sent, and in various embodiments sends a corresponding control command or signal to the computer 114 to adjust (increase or decreases) the QoS, such as the bandwidth used across the communication link 108.

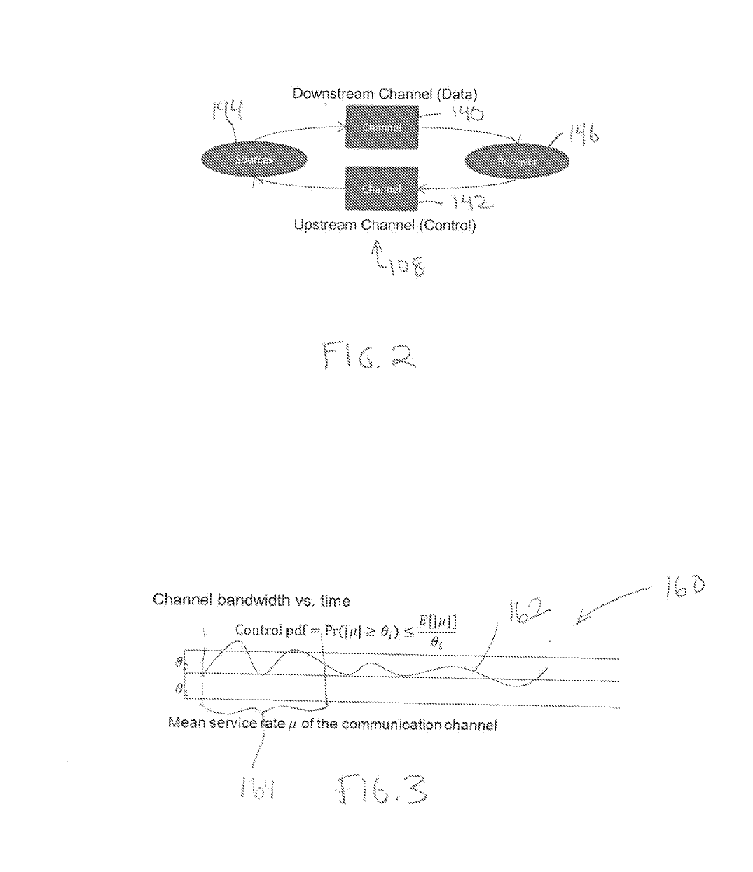

In one embodiment, as illustrated in FIG. 2, the communication link 108 may be formed from a downstream channel 140 and an upstream channel 142 that define a data channel and a control channel, respectively. The downstream channel 140 is configured to communicate or transfer data (e.g., data packets) from a plurality of data sources 144 (which may be from the medical devices 120 shown in FIG. 1) to a receiver 146 (which may be embodied as the specialist transceiver 106 shown in FIG. 1).

In operation, the channel bandwidth monitored by the channel capacity monitoring unit 150 (shown in FIG. 1) uses the sliding window to determine whether a channel bandwidth signal exceeds a defined threshold. It should be noted that the threshold value may be fixed or defined in some embodiments, while in other embodiments, may be a dynamically computed parameter. The channel bandwidth in some embodiments is determined from a signal described by a random process as described in more detail herein. In one embodiment, when the channel bandwidth signal exceeds the defined threshold, a control message is transmitted by the channel capacity monitoring unit 150 using the receiver 146 back to the sources 144 indicating adjustments to the transmission rates for the medical devices 120 that are the sources 144 of the data (e.g., medical monitoring data).

In various embodiments, using the Markov Inequality, it is known that the rate of the control messages (Control pdf) will be no greater than the mean of the downstream bandwidth divided by the selected or defined threshold, which may be defined as follows:

$$Control pdf = Pr ( \vert μ \vert \geq {θ}_{i} ) \leq \frac{E [ \vert μ \vert ]}{{θ}_{i}} \qquad Eq . 1$$

where E is an expected value, μ is the average bandwidth or rate of data packets and θ is the defined threshold.

Markov's Inequality generally gives an upper bound for the probability that a non-negative function of a random variable is greater than or equal to some positive constant. Markov's Inequality relates probabilities to expectations, and provides bounds for the cumulative distribution function of a random variable.

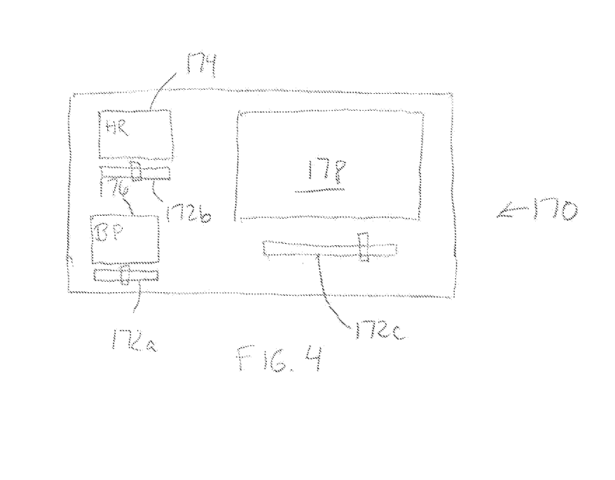

Using Equation 1, the probability (Pr) of exceeding the threshold θ may be determined by the Markov Inequality. For example, the Markov Inequality determines what the ratio of the upstream to downstream mean values should be relative to the cutoff values θ, which in the graph of FIG. 3 is defined by θ₂ and θ₁, setting upper and lower cutoff values respectively. In FIG. 3, the curve 162 represents information from the medical devices 120 corresponding to a mean service rate μ of the communication channel. The curve 162 is a plot of channel bandwidth over time. Accordingly, in various embodiments, as the signal represented by the curve 162 corresponding to the channel bandwidth signal within the sliding window 164 increases in variance (e.g., less smooth), the control packets communicated to the medical devices 120 to adjust the transmission rate thereof also increases.

In one embodiment, in order to minimize or reduce channel congestion, and accordingly feedback latency, on the upstream channel 142, the rate of control messages is defined to not exceed the following:

E[μUp]/E[μDown] Eq. 2

which is derived from:

E[μUp]/θ(control packets/second)==E[μDown](packets/second],

θ==EμUp]/E[μDown] Eq. 3

where E is an expected value, μUp is the average bandwidth or rate of data packets on the upstream channel, μDown is the average bandwidth or rate of data packets on the downstream channel, and θ is the defined threshold.

Thus, according to Equations 2 and 3, θ increases as the downlink rate decreases, causing the control message transfer rate to decrease, and vice versa.

The sliding window of channel bandwidth samples is maintained by collecting samples either from the channel itself or at the receiver 146. It should be noted that if the channel bandwidth is estimated by measuring received packets, then this is an approximation because there is a small transmission delay and the combined sources 144 may not be utilizing the entire new channel bandwidth if the bandwidth increases. However, various embodiments, assume the approximation is correct.

By adjusting the rate of communicating control messages based on θ, the feedback response is increased or maximized when the response occurs, although the response will occur less often as the downlink channel becomes smaller relative to the uplink channel. Accordingly, the sources 144 adjust more rapidly when the upstream variation is low and more slowly as the uplink variation becomes large. This adjustment of the sources 144 reduces or minimizes large sudden changes in QoS.

By using various embodiments, the rate at which medical data is transmitted is adjusted or optimized to increase or maximize a QoS to the remote user (e.g., specialist).

It should be noted that the threshold values θ may be initially set or arbitrarily defined and also adjusted. The setting of the threshold value may be a one time setting or may be dynamically adjusted. The threshold values generally define when control packets are communicated back to the sources 144 to adjust the transmission rate for data from the sources, such as medical data from the medical devices 120. Thus, various embodiments use a certain amount of the channel bandwidth while achieving a certain level of quality (e.g., QoS).

It should be noted that each type of data may have a corresponding rate distortion curve defining bandwidth versus quality. Accordingly, in various embodiments, the transmission rate of the plurality of sources 144 may be concurrently adjusted to increase or optimize the medical data communicated from the sources 144 while staying within the channel constraints. For example, the constrained channel in various embodiments may not be able to maintain transmission of all data, for example, video, blood pressure measurements and heart rate measurements with fidelity. As an example, the video may initially appear blurry with the fine detail missing or indiscernible, while the blood pressure and heart rate measurements are communicated without any reduction in quality. However, continuing with the example, if the specialist wants to stress the patient (e.g., asks the patient to jump up and down), the specialist may want to see if the patient is breathing harder. Thus, a higher resolution image of the patient's face may be desired, while heart rate information is not sent or sent at increased time intervals.

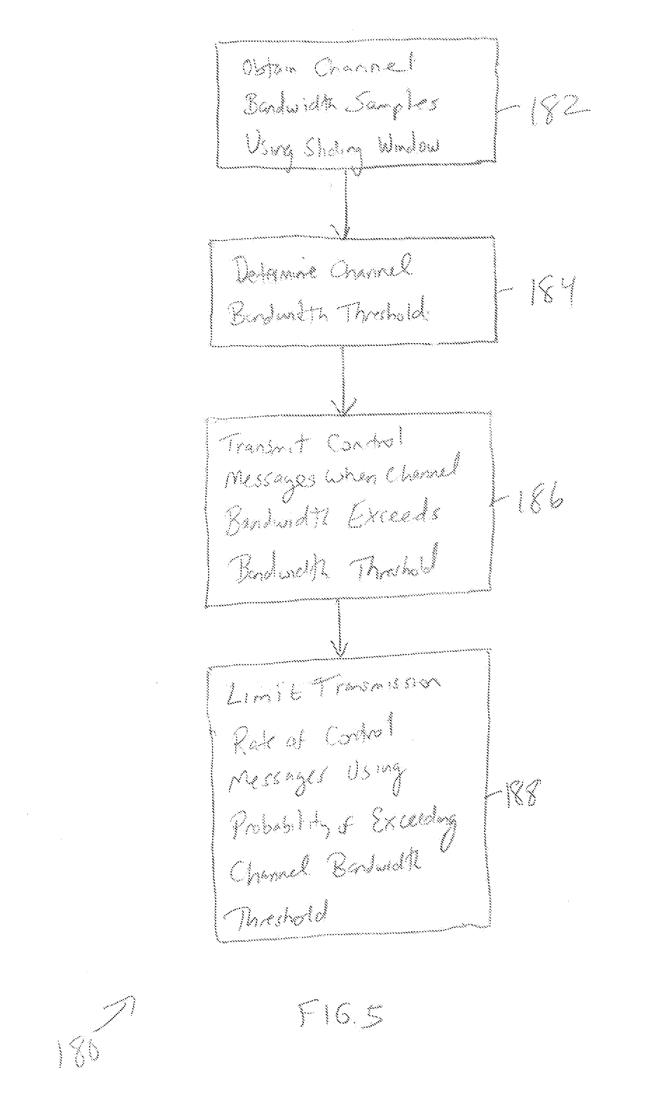

In various embodiment, a user interface 170 as shown in FIG. 4 may be provided to allow a user (e.g., a specialist) to adjust a quality level of medical data communicated from, for example, the medical device 120 to the second location 112 where the user is located. In accordance with various embodiments, as the quality level for one or more of the transmitted medical data is adjusted, the rate of feedback packets (e.g., control signals based on the quality level adjustments) is also adjusted as described herein, as the feedback packets also use bandwidth of the channels.

As illustrated in FIG. 4, user interface elements, which may be slider bars 172 may be displayed as part of the user interface 170. A separate slider bar 170 may be provided for each type of information communicated and displayed, which in the illustrated embodiment is heart rate (HR) information 174, blood pressure (BP) information 176 and images 178 (e.g., video), having corresponding slider bars 172a, 172b and 172c. It should be noted that if different or additional information is displayed, different or additional slider bars 172 are provided. Additionally, the relative scales for the slider bars 172 may be different, such as a larger or higher level of granularity for the images 178. It also should be noted that the slider bars 172 may provide continuous or incremental adjustments along the slider bars 172.

Using various embodiments, as the slider bars 172 are adjusted, for example, moved up (to the right in FIG. 4) to increase the quality of the transmission of the corresponding information, with the bandwidth for the communication of that data increased, which may result in a decrease in the bandwidth for the communication of the other data, such that the other slider bars 172 may be automatically adjusted down (to the left in FIG. 4). Thus, by adjusting the slider bars 172 the user may effectively adjust the thresholds θ or rate distribution curve setting.

Various embodiments provide a method 180 as shown in FIG. 5 for channel capacity change detection to control a rate of transmission of control packets that are to adjust the transmission rate of data from one or more sources (e.g., medical devices). The method 180 includes obtaining channel bandwidth samples using a sliding time window at 182. As described in more detail herein, the channel bandwidth samples may be determined by measuring received data packets, such as to determine a mean service rate of the communication channel.

The method 180 also includes determining a channel bandwidth threshold at 184. The threshold bandwidth may be a range or an upper limit, which may be set one time (e.g., Information Technology (IT) setting) and/or dynamically changed.

The method 180 further includes transmitting control messages at 186 when the channel bandwidth exceeds the bandwidth threshold. For example, if a determination is made using the bandwidth samples that the channel bandwidth exceeds the bandwidth threshold, a control message is transmitted to the sources of the data transmission (e.g., medical devices) to adjust the rate of transmission of the data from the sources.

The method 180 additionally includes limiting the transmission rate of the control messages using the probability of exceeding the channel bandwidth threshold. As described in more detail herein, the Markov Inequality may be used to determine what the ratio of the upstream and downstream mean values for the transmission rates should be to the cutoff values defined by the bandwidth threshold. The Markov Inequality generally allows for a determination of the probability that the transmission of the control messages will cause the channel to exceed the bandwidth threshold. Accordingly, to reduce or minimize congestion, and thus feedback latency, on the upstream channel, the rate of control messages on the upstream channel is limited based on the predicted or expected values. Thus, θ increases as the downlink rate decreases, causing the rate of transmission of the control messages to decrease.

Thus, various embodiments change the rate at which data is sent to change the bandwidth usage to position the transmission rate on a rate distortion curve. However, because the channel is divided proportionally, but the total channel bandwidth availability changes, the rate of transmission may be reduced to maintain the same proportionality of the channel, for example, by reducing the number of control packet communicated. Thus, various embodiments may provide rate control matching, such that more control packets may be communicated when the channel bandwidth has increased variance to thereby control the proportions, and less control packets communicated when the change is smoother.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, flash drive, jump drive, USB drive and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the described subject matter without departing from their scope. While the dimensions and types of materials described herein are intended to define the parameters of the various embodiments, the embodiments are by no means limiting and are exemplary embodiments. Many other embodiments will be apparent to one of ordinary skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable one of ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for controlling transmission of medical data, the method comprising:
monitoring a bandwidth over time of a channel communicatively coupling a plurality of medical devices at a first location with a second location remote from the first location;
determining when a channel bandwidth of the channel exceeds a defined threshold value using the monitored bandwidth;
transmitting control signals from the second location to the plurality of devices at the first location to adjust a transmission rate of medical data from the plurality of medical devices to the second location; and
limiting a rate of transmission of the control signals to the plurality of medical devices based on a probability that the transmission of the control signals causes the channel bandwidth to exceed the defined threshold value.

2. The method of claim 1, further comprising using a Markov Inequality to determine the probability that the transmission of the control signals causes the channel bandwidth to exceed the defined threshold value.

3. The method of claim 1, wherein limiting the rate of transmission of the control signals is defined by:
E[μUp]/E[μDown]
which is derived from:
E[μUp]/θ(control packets/second)==E[μDown](packets/second],
θ==EμUp]/E[μDown]
where E is an expected value, μUp is the average bandwidth or rate of data packets on the upstream channel, μDown is the average bandwidth or rate of data packets on the downstream channel, and θ is the defined threshold.

4. The method of claim 1, further comprising defining a transmission rate of the control signals as:
$$Control pdf = Pr ( \vert μ \vert \geq {θ}_{i} ) \leq \frac{E [ \vert μ \vert ]}{{θ}_{i}}$$
where the where E is an expected value, μ is the average bandwidth or rate of data packets and θ is the defined threshold.

5. The method of claim 1, wherein monitoring the bandwidth of the channel over time comprises monitoring a receive rate of data packets over a sliding time window.

6. The method of claim 1, further comprising receiving one or more user inputs adjusting a slider bar setting of a user interface to adjust a quality level of data communicated over the channel, wherein the user input causes a change in the transmission rate of the medical data.

7. The method of claim 1, wherein the channel comprises a constrained channel having a randomly changing data transmission capacity.

8. The method of claim 1, wherein the medical data comprises different types of data and further comprising providing corresponding rate distortion curves for the different types of data defining bandwidth versus quality

9. A medical data communication system comprising:
a plurality of medical devices at one location configured to acquire medical data for a patient;
a transceiver coupled to the plurality of medical devices;
a workstation at a location remote from the location of the plurality of medical devices;
a transceiver coupled to the workstation, the transceivers coupled to the plurality of medical devices and the workstation forming a communication link therebetween; and
a channel capacity monitoring unit at the location of the workstation, the channel capacity monitoring unit configured to monitor a bandwidth over time of a channel of the communication link, determine when a channel bandwidth of the communication link exceeds a defined threshold value using the monitored bandwidth, transmit control signals to the plurality devices to adjust a transmission rate of medical data from the plurality of medical devices, and limit a rate of transmission of the control signals to the plurality of medical devices based on a probability that the transmission of the control signals causes the bandwidth of the communication link to exceed the defined threshold value.

10. The medical data communication system of claim 9, wherein the channel capacity monitoring unit is further configured to use a Markov Inequality to determine the probability that the transmission of the control signals causes the bandwidth of the communication link to exceed the defined threshold value.

11. The medical data communication system of claim 9, wherein the channel capacity monitoring unit is further configured to limit the rate of transmission of the control signals using:
E[μUp]/E[μDown]
which is derived from:
E[μUp]/θ(control packets/second)==E[μDown](packets/second],
θ==EμUp]/E[μDown]
where E is an expected value, μUp is the average bandwidth or rate of data packets on the upstream channel, μDown is the average bandwidth or rate of data packets on the downstream channel, and θ is the defined threshold.

12. The medical data communication system of claim 9, wherein the channel capacity monitoring unit is further configured to define a transmission rate of the control signals as:
$$Control pdf = Pr ( \vert μ \vert \geq {θ}_{i} ) \leq \frac{E [ \vert μ \vert ]}{{θ}_{i}}$$
where the where E is an expected value, μ is the average bandwidth or rate of data packets and θ is the defined threshold.

13. The medical data communication system of claim 9, wherein the channel capacity monitoring unit is further configured monitor a receive rate of data packets over a sliding time window.

14. The medical data communication system of claim 9, wherein the workstation comprises a user interface and the channel capacity monitoring unit is further configured to receive one or more user inputs adjusting a slider bar setting of the user interface to adjust a quality level of data communicated over the communication link, wherein the user input causes a change in the transmission rate of the medical data.

15. The medical data communication system of claim 9, wherein the communication link comprises a constrained channel having a randomly changing data transmission capacity.

16. A non-transitory computer readable storage medium for controlling the communication of medical data over a channel using a processor, the non-transitory computer readable storage medium including instructions to command the processor to:
monitor a bandwidth over time of a channel communicatively coupling a plurality of medical devices at a first location with a second location remote from the first location;
determine when a channel bandwidth of the channel exceeds a defined threshold value using the monitored bandwidth;
transmit control signals from the second location to the plurality devices at the first location to adjust a transmission rate of medical data from the plurality of medical devices to the second location; and
limit a rate of transmission of the control signals to the plurality of medical devices based on a probability that the transmission of the control signals causes the channel bandwidth to exceed the defined threshold value.

17. The non-transitory computer readable storage medium of claim 16, wherein the instructions command the processor use a Markov Inequality to determine the probability that the transmission of the control signals causes the channel bandwidth to exceed the defined threshold value.

18. The non-transitory computer readable storage medium of claim 16, wherein the instructions command the processor to limit the rate of transmission of the control signals using:
E[μUp]/E[μDown]
which is derived from:
E[μUp]/θ(control packets/second)==E[μDown](packets/second],
θ==EμUp]/E[μDown]
where E is an expected value, μUp is the average bandwidth or rate of data packets on the upstream channel, μDown is the average bandwidth or rate of data packets on the downstream channel, and θ is the defined threshold.

19. The non-transitory computer readable storage medium of claim 16, wherein the instructions command the processor to define a transmission rate of the control signals as:
$$Control pdf = Pr ( \vert μ \vert \geq {θ}_{i} ) \leq \frac{E [ \vert μ \vert ]}{{θ}_{i}}$$
where the where E is an expected value, μ is the average bandwidth or rate of data packets and θ is the defined threshold.

20. The non-transitory computer readable storage medium of claim 16, wherein the instructions command the processor to receive one or more user inputs adjusting a slider bar setting of a user interface to adjust a quality level of data communicated over the channel, wherein the user input causes a change in the transmission rate of the medical data.

21. The non-transitory computer readable storage medium of claim 16, wherein the channel comprises a constrained channel having a randomly changing data transmission capacity.
